(19) United States
(12) Patent Application Publication
(10) Pub. No.: US 2013/0116192 A1
(43) Pub. Date: May 9, 2013

(54) N-(AMINOACYL)-AMINO COMPOUND

(71) Applicant: Gosbert Weth, Bad Kissingen (DE)

(72) Inventor: Gosbert Weth, Bad Kissingen (DE)

(21) Appl. No.: 13/638,422

(22) PCT Filed: Mar. 30, 2011
(86) PCT No.: PCT/IB2011/001902

§ 371 Date: Nov. 8, 2012

(30) Foreign Application Priority Data
Mar. 31, 2010 (DE) 10 2010 013 587.9

(51) Int. Cl. A61K 38/05; C07K 5/06

(52) U.S. Cl. 514/17.8; 562/561; 514/564; 514/565; 514/21.91; 562/556; 548/536; 514/423; 546/245; 514/315; 548/201; 514/365; 546/193; 514/318; 514/276; 514/52; 514/249; 514/167; 436/113

(57) ABSTRACT

N-(aminoacyl)-amino compound, represented by the following formula

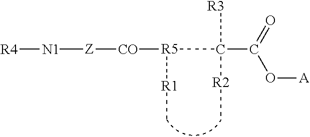

Wherein R1 denotes hydrogen, low alkyl or carbonyl, and N1 denotes an NH group and
R2 denotes hydrogen or low alkylphenyl or aralkyl or imidazoalkyl or indolylalkyl,
R1 and R2 together may complete a pyrrolidine or piperidine or thiazolidine ring and
R3 denotes hydrogen or methyl or low alkyl and R4 denotes hydrogen or alkyl or the group remaining on exclusion of R4 from the formula and
Z is a straight chain or branched alkylene, which may contain up to 3 carbon atoms. and R5 is nitrogen or sulphur or oxygen or salts thereof and ester compounds, characterised in that A is an ester or amino acid or alternatively sodium or a potassium salt of arginate and/or of ornithate and/or of aspharaginate.

The invention relates to an N-(aminoacyl)-amino compound according to the definition of claim 1.

An essential component of this substance are N-(aminoacyl)-amino acids, which are known per se. Thus, this group includes, for example, the ACE blockers (angiotensin-converting enzyme), which are widely used as antihypertensives. It is known that high blood pressure is a significant risk factor for Alzheimer's disease, very often known colloquially just as “Alzheimer's”, at which an important focus of this invention is directed.

Alzheimer's disease is a neurodegenerative illness that, in its most frequent form, occurs in people above the age of 65 and in 2005 affected approximately 60 percent of the approximately 24 million dementia sufferers. (C. P. Ferri, M. Prince, C. Brayne et al.: Global prevalence of dementia: a Delphi consensus study. In: Lancet. 366, Nr. 9503, 2005, S. 2112-7. doi:10.1016/80140-6736(05)67889-0. PMID 16360788)

Due to demographic development in the Western industrial nations, with increasingly ageing citizens, the prevalence of Alzheimer's is also increasing. Below the age of 65, only about 2% are affective; among 70-year-olds it is already 3% and among 75-year-olds 6% and of 85-year-olds about 20% show symptoms of the illness. Above the age of 85, the proportion of those affected decreases again, since those previously affected only rarely reach this age.

In Germany, over one million people suffer from a dementia illness, 700 000 of those from Alzheimer's disease.

Each year, approximately 200 000 new dementia illnesses are diagnosed, of which about 120 000 are of the Alzheimer type.

In 2007, about 29 million people worldwide were affected by Alzheimer's disease. According to population forecasts by the United Nations, this number will increase to about 106 million patients by 2050; on average, there will then be one Alzheimer's patient per 85 people.

The characteristic symptom is an increasing impairment of cognitive performance, which in generally associated with a decrease of daily activities, with behavioural abnormalities and neuropsychological symptoms. To check the current state of cognitive loss simply and quickly, the “clock drawing test” has proven itself, in which the patent is verbally set the task of graphically describing a clock face. The result is essential for quantifying the illness.

The analysis of chemical processes in the brain of Alzheimer's patients shows that, in the course of the illness, the neurotransmitter acetylcholine is no longer produced in sufficient quantities, inter alia by a reduction of the enzyme choline acetyl transferase occurring in the nucleus basalis of Meynert, which catalyzes the joining of acetyl-CoA and choline, which leads to a general weakening of the performance of the brain. The most spectacular symptom, however, is that the brain mass progressively decreases due to the dying of neurons, which is also termed brain atrophy.

Even many years before the symptoms become clinically visible, senile plaques form in the brains of those affected. They are protein deposits, which consist of incorrectly folded amyloid beta (Aβ) peptides. The Aβ peptide results from a precursor protein, the amyloid-precursor protein (APP), which is an integral membrane protein. The greatest proportion of this protein projects out of the cell and is located in the extracellular matrix, while only a minor proportion is disposed within the intracellular matrix. The Aβ peptide is a type 1 transmembrane protein, whose amino terminal group is on the outside of the cell, while its carboxyl terminal group can be found within the cell.

These protein deposits in the brain of Alzheimer patients—often called amyloid plaques for short—are very characteristic of the illness. This is the basis of the so-called “amyloid” hypothesis of Alzheimer's disease. The plaques form as extracellular amyloid deposits and occur when the proteins formed in the cells are released. It can be observed that a cell dies when the amyloid proteins can no longer be released from the cell.

Some of the therapy processes that are currently being investigated therefore aim at reducing this plaque. The positive effect of the medications currently used, however, is very low. Their effect is only symptomatic and as a result of taking them, admission to a home can only be postponed by half a year. In addition, their application is restricted by considerable side effects.

Until now, however, it is unclear if the cell loss can be stabilized at all by reducing the plaques. Although plaques also have toxic effects, these are rather to be classified as a by-product. In the current state of research, it is very doubtful whether the nerve cell loss can be correlated at all with the amyloid hypothesis.

It would be much more sensible to combat the formation of amyloid beta peptides in the cell already at a very early stage, and not only when the plaques have already occurred. New research therefore gives reason to assume that the actual causal processes take place in the nerve cells themselves. (Prof. Dr. Thomas Beyer, University of Göttingen, Interview with arte-tv, Apr. 7, 2008)

At the same time as the plaques, fibrillar deposits occur, which accumulate in the neurons, so-called neurofibrils which are also a clear indication of the illness. These intracellular neurofibril bundles consist of a tau protein, which aggregates into fibrils if it is normally phosphorylated, that is to say has phosphoric acid residues added to it (“hyperphosphorylation”). Until now it has still not been explained whether this tau phosphorylation is only secondary in nature or whether it is actually pathogenic.

The latest investigations in patients with dementia symptoms that were actually clearly attributed to Alzheimer's disease, also clearly indicate that the causality of the dementia symptoms is to be sought intracellularly. According to these results, however, it is not the neurofibrils that are the actual triggers but rather ammonia and ammonia compounds. Also in the case of patients having the same symptoms, though caused by kidney failure, ammonia or ammonia compounds have been observed in clearly excessive amounts.

Since the causes of Alzheimer's disease are currently not clearly known, there are, in the prior art, also experiments on the therapy of this illness with an extremely wide variety of active substances.

In the current state of knowledge, it is generally undisputed that the three main risk factors for Alzheimer's disease are known, namely diabetes, overweight and high blood pressure.

The relationship between high blood pressure and Alzheimer's disease is provided by a study by Prof. Jan Staessen, University of Leuven, published in The Archives of Internal Medicine, edition Oct. 14, 2002: For almost four years, almost 3 000 patients were observed in the framework of a European study on high blood pressure. In the first part of the study, they had received either antihypertensives or a dummy drug. Only in the second stage were all participants treated with antihypertensive medications. Of the experimental participants that had obtained effective medication from the start, somewhat fewer than half, as in the placebo group, became ill with dementia, with Alzheimer's disease being the most frequent dementia illness by far.

A medication that is known and proven to be effective against high blood pressure is described by U.S. Pat. No. 5,589,499 as N-(aminoacyl)-amino acid. Because of its dopaminergic effect, it leads both peripherally and centrally to an increase in blood flow. This effect can also be proven cerebrally, in which the dopaminergic effect is determined by varying of the neurotransmitter. This cerebral effective is also plausible in that dopaminergic substances, such as, for example, dopa, are also used successfully in Parkinson's patients.

The effect achieved thereby in the Alzheimer's disease is noticeable and reduces suffering, however does not lead to a freedom from complaints and not at all to a regain of the brain capabilities that were formerly available.

Against this background, it is the object of the invention to find an active substance that quite significantly reduces the effects of Alzheimer's disease and attenuates or even entirely avoids the cell loss.

As a solution, the invention presents the active substances N-(aminoacyl)-amino ester or an N-(aminoacyl)-amino group that is bound to an amino acid according to the description in claim 1 and N-(aminoacyl)-amino salt according to the description in claim 7.

It is the basic concept of this invention, to introduce, through the blood-brain barriers into the brain cells, N-(aminoacyl)-amino acid, which has been proven to have a blood-flow promoting effect and which for that reason alone is effective against Alzheimer's disease, which additionally also additionally has an ammonia removing, that is to say a toxin-releasing, effect. Overcoming the blood-brain barrier is achieved by esterification or salt formation of the N-(aminoacyl)-amino acid or the chemical addition of a further amino acid. Both the ester and the salt or the compound with a further amino group serve primarily for the transport of the N-(aminoacyl)-amino acid to the desired effect location and are themselves only involved marginally in the action process.

The active substance transport according to the invention is obtained by an extremely wide variety of esters. It is particularly intensive if the ester is an asparaginate ester and/or an arginine ester and/or an ornithate ester.

Since these three esters have proven to be particularly effective in the sense of the invention, the advantages of the invention are to be explained below with the example of the three chosen esters, and it is to be illustrated how they help to overcome the blood-brain barriers according to the gist of the invention.

In the blood-brain barrier, a special form of diffusion through the cell membrane of the endothelia is “facilitated diffusion.” For molecules that are too large, there is a special transport system in the cell membrane: so-called carrier-mediated transport.

The special SLC7 transporter transports cationic amino acids, inter alia arginine and ornithine. SLC6 is responsible for the transport of the neurotransmitters dopamine and noradrenaline. Another carrier transports D-aspartic acid. In the brain, it is a precursor of N-methyl-D-aspartate (NMDA) and influences the secretion of various hormones such as luteinizing hormone, testosterone or oxytocin. L-aspartic acid, together with L-glutamic acid, is among the stimulating amino acids.

The presence of the so-called carriers for the transport of the three substances, whose esters are characteristic of the invention, makes clear how effective these substances are for N-(aminoacyl)-amino acid for overcoming the blood-brain barriers. For the three characteristic substances special transport substances are thus available in the blood-brain barrier, which enable the path through this barrier. By the fact that these three substances in turn carry the N-(aminoacyl)-amino acid with them, it is also possible for this substance to pass through the blood-brain barrier, which in a normal case is actually blocked for it. The esterification by these three substances that are capable of passage is thus a “free ride” for the actual active substance.

Below, it is to be explained what properties the three characterizing substances ornithine, arginine and asparagine have, which are supposed also to have positive effects in the meaning of the object of the invention.

Ornithine (from the Greek: ornis, bird) is a basic, non-proteinogenic α-amino acid. It acts as a carrier substance for example in the urea cycle.

Ornithine was prepared for the first time by Jaffe in 1877 from fowl excrement. The industrial production of ornithine according to the prior art is performed by hydrolysis of L-arginine in an alkaline medium.

L-arginine is a proteinogenic α-amino acid. The name is derived from the Latin word argentum (silver), since the amino acid was at first able to be isolated as a silver salt. This amino has the highest proportion by mass of nitrogen of all proteinogenic amino acids. Arginine predominantly exists as an “inner salt” or zwitterion, the formation of which can be explained by the fact that the proton of the carboxyl group migrates to the guanodino residue, which is more basic than the α-amino group.

For humans, it is semi-essential. L-arginine is a metabolite of the urea cycle, in which the ammonia that occurs on degradation of nitrogen compounds (for example amino acids) is converted into urea. L-arginine hydrochloride, too, is appropriate for treating a high ammonia content in the blood (hyperammonaemia) caused by a severe congenital metabolic defect. This effect, which is known per se, is with some probability also part of the effect of the substance according to the invention, N-(aminoacyl)-amino arginate ester, for treating a toxic excess of ammonia and ammonia compounds in the cell.

L-asparagine is a proteinogenic α-amino acid. It is a derivative of the acidic amino acid L-aspartic acid. Instead of the γ-carboxyl group, it carries an amide group, occurs as a betaine (inner salt) at the isoelectric point (pH) and is a hydrophilic amino acid. Asparagine predominantly exists as an “inner salt” or zwitterion, the formation of which can be explained by the fact that the proton of the carboxyl group migrates to the lone electron pair of the nitrogen atom of the amino group.

As already mentioned, an esterification of the N-(aminoacyl)-amino acid with these substances is thus an effective “transport means” in the blood-brain barrier for coupling to the “carriers” provided there, and in this manner reaching the brain, in order to unfold its beneficial effect in the battle again an increase of the level of ammonia or ammonia compounds in the battle against Alzheimer's disease.

A further, interesting alternative of the N-(aminoacyl)-amino compound according to the invention is to add an amino acid as group A. All essential amino acids have the positive property that they can overcome the blood-brain barrier. If another substance is attached thereto, which could not pass through the blood-brain barrier on its own, the accessibility is hardly changed. The amino acid thus also acts as a carrier for the route through the blood-brain barrier.

After overcoming the blood-brain barrier, the amino acid and the substance group attached thereto are separated from one another again by hydrolysis. Then the substance group described in claim 1 without A—the N-(aminoacyl)amino—acts.

Of the amino acids provided as carriers, all acidic amino acids have the disadvantage that they disadvantageously shift the pH in the mitochondria into the acid range. For this reason, the invention prefers basic amino acids.

In tests, the following four basic amino acids, namely asparagine aspartate, arginine aspartate, arginine lysine and cysteine have proven especially suitable and are therefore preferred by the invention.

It is to be explained in greater detail below why ammonia, ammonium, and ammonium derivatives are cell toxins and how they act on brain cells.

It is assumed that at least a significant—if not even the most important—cause of Alzheimer's disease is an overloading of the brain cells with ammonia or ammonia compounds. That is plausible because it is undisputed that relatively large amounts of ammonia are toxic in the body. It is known that, for the transfer of substantial amounts of ammonia into the blood, which increases the blood level of NH₄+ to over 35 μmol, central nervous symptoms, such as tremors of the hands, speech and visual disturbances and confusion, as far as coma and death, occur. The pathophysiological mechanisms that take place here, however, have not yet been clearly explained.

Ammonia—a chemical compound of nitrogen and hydrogen with the molecular formula NH₃—or ammonia derivatives are always present in humans and vertebrates, since, biologically, ammonia has an important function as an intermediate in the formation and degradation of amino acids. All vertebrates therefore have chemical processes for converting ammonia into the non-toxic urea. In the event of a failure of these processes, ammonia, ammonium or ammonia compounds occur in large amounts and then—as mentioned above—have either a toxic or even highly toxic effect.

Ammonia appears principally to damage astrocytes in the brain. Astrocytes, also known as “star cells”, “spider cells” or “glial cells”, are cells that are branched like stars or spiders, whose continuations form limiting membranes to the brain surface (or pia mater) and the blood vessels. Astrocytes feed the neurons via contacts to blood vessels and are also essentially involved in the fluid regulation in the brain.

Astrocytes also form the “membrana limitans glialis perivascularis”, which induces and maintains the endothelial blood-brain barrier. The blood-brain barrier is a physiological barrier between the blood circulation and the central nervous system (CNS) that is present in all terrestrial vertebrates (tetrapoda). It serves for maintaining the milieu conditions (homoeostasis) in the brain and separating them from those of the blood. The principal component of this barrier is endothelial cells, which are tightly linked to one another via so-called “tight junctions”, and line the capillary blood vessels towards the blood.

The blood-brain barrier protects the brain against pathogens, toxins and messengers circulating in the blood. It represents a highly selective filter, via which the nutrients required by the brain are supplied and the resulting metabolic products are eliminated. Not only the supply but also the elimination are ensured by a range of special transport processes.

That is essential, since, due to the high energy demand—in comparison to other organs—of the brain, excessive amounts of metabolic degradation products are produced, which must be eliminated again via the blood-brain barrier (S. Ohtsuki: New aspects of the blood-brain barrier transporters: its physiological roles in the central nervous system. In: Biol Pharm Bull. 27, 2004, pp. 1489-1496. PMID 15467183)

However, this protective function of the brain makes drug treatment of a wide variety of neurological illnesses difficult, since a very large number of active substances can also not pass through the blood-brain barrier, and therefore overcoming the blood-brain barrier is a highly topical research area to allow these illnesses to be treated, too.

It is an important function of the astrocytes in the blood-brain barrier that they ensure the potassium household is maintained. The potassium ions liberated in the nerve cells during the saltatory conduction are absorbed into the glial cells predominantly by a high potassium conductivity and partly also by K⁺ and Cl⁻ cotransporters.

Another explanation for the neurotoxic effect of ammonium is the similarity of ammonium to potassium. Due to the exchange of potassium with ammonium, disturbances of the activity of the NMDA receptor occur and as a consequence an increased calcium flow into the nerve cells, which causes the death of these cells. (D. J. Randall, T. K. N. Tsui: Ammonia toxicity in fish. In: Marine Pollution Bulletin. 2002, 45, pp. 17-23 (doi:10.1016/S0025-326X(02)00227-8, PMID 12398363).)

Poisoning with ammonia leads to a demonstrable damage to the NMDA receptor. The ammonia load decreases the membrane voltage of the nerve cells from its normal 80 to 100 my to up to 20 mV. As a result, the NMDA receptor of the nerve cells is changed such that magnesium emerges and the cells with inflowing calcium ions are overloaded such that they enter into a stress situation, in which they can be permanently damaged by NO* radicals in toxically increased concentration and by oxygen radicals and even die off.

An increase in the conductivity of the NMDA receptor is, according to current teaching, categorized as essential for the induction of synaptic plasticity. It is thus a molecular mechanism for learning and memory. Thereby, the NMDA receptors of particularly very frequently used synaptic pathways are unblocked by the constant depolarization of the postsynaptic membrane, as a result of which their conductivity is increased with respect to other circuit patterns. In this manner, particular “pathways” are opened, which is an essential process of learning. As a result, it is clear that a pathological faulty control of the NMDA receptors in the context of sicknesses can arise, which originates from the brain.

Another confirmation of the effect of ammonia as cell toxin for nerve and muscle cells is contained in G. Halwachs-Baumann: La-bormedizin: Klinik—Praxis—Fallbeispiele, Springer 2006, ISBN 978-3-21125291-8 on page 96.

Almost all biological membranes are permeable to ammonia because of the low size of the molecule and its lipid solubility. (G. F. Fuhrmann: Toxikologie für Naturwissenschaftler: Einführung in die theoretische and spezielle Toxikologie. Vieweg+Teubner Verlag, 2006, ISBN 978-3-83510024-4, p. 53, p. 349)

The encephalotoxic effect is also partly linked to an increased glutamine level in the brain (J. Hallbach: Klinische Chemie für den Einstieg. 2. Auflage, Georg Thieme Verlag, 2006, ISBN 978-3-13106342-7, S. 207) and in association with the formation of reactive oxygen species (J. E. O'Connor, B. F. Kimler, M. Costell, and J. Vina: Ammonia Cytotoxicity Involves Mitochondrial Disfunction, Impairment Of Lipid Metabolism And Oxidative Stress. Dpt. of Biochemistry, University of Valencia, Valencia, Spain; Dpt. of Radiation Biology, Kansas University Medical Center, Kansas City, Kans.; Institute de Investigaciones Citolögicas, Valencia, Spain).

NH₃ and NH₄—OH derivatives are powerful cell toxins and, in particular in the case of an overloading by H+ ions, lead to a disruption of the metabolism on the cell membranes and in the mitochondria. As a result, the number of mitochondria is reduced and the remaining mitochondria are subsequently damaged. The result is a deficit in the energy-rich phosphate ATP. Consequently, the membrane function is restricted and the membrane potential is reduced to values below 70 mV.

The cell potential reduced by toxic molecules—for example NH₃—can be normalized by a complementary pulse therapy, that is to say to values of 80 mV to 100 mV. At this normal value of the cell potential, NH₃ can be directly flushed out of the cell again with the aid of the substances according to the invention.

The application of electromagnetic impulses, which make the cell membrane permeable for NH₃ at all, has led to the discovery that NH₃ is the decisive cause of the dying off of brain cells.

Through the complementary administration of electromagnetic pulses, the potential of the cell membranes of the mitochondria is also normalized.

If the cell potential has normal values of about 80 mV to 100 mV, the function of the respiratory chain—the ATP function—is no longer disrupted, which is the case with NH₃ overloading. The glutamine overloading disappears.

Furthermore, the NH₃ formation leads to an activation of the β and γ secretases, which are responsible for forming the plaque. The overloading by plaques leads to an inflammation reaction, and also the toxic ammonia derivative and its ammonium derivatives. This chronic inflammation process accelerates the formation of pathological proteins such as APP and tau filaments.

Now that the inventive principle so far, and for this purpose also the effect of ammonia and ammonia compounds as cell toxin has been explained, various embodiments are presented below: detailed series of experiments have shown that each of the three esters in itself also shows an effect according to the invention. However, it proved even more effective when the N-(aminoacyl)-amino acid with all three characterising substances were transformed into esters and the actual active substance is a mixture of these three esters. A mixture is preferred in which the proportion of the arginate ester is greater than the proportion of the ornithate ester or of the asparaginate ester. It proved very effective to increase the proportion of arginate ester to at least 50%. A further increase could be obtained if the proportions of ornithate ester and asparaginate ester were approximately the same.

A very important application field of the substance according to the invention is the use for manufacturing pharmaceutical preparations and medicaments. According to the object of the invention, the treatment or prevention of toxic loadings by ammonia, ammonium and/or ammonia compounds is paramount. As already mentioned, one of the most interesting applications, which with some probability can be attributed to a toxic overloading with ammonia or ammonia compounds, is Alzheimer's disease, with its above-mentioned devastating consequences for a large proportion of the elder population.

The effectiveness of the substance according to the invention could be proven, inter alia, on a group of 20 successfully treated patients. All patients suffered from clearly marked symptoms of dementia. In order to have a comparable indicator or the degree on illness, the so-called “clock drawing test” is a widely accepted test. This test showed a significant different for the aforementioned patient group before and after the treatment. A typical example is reproduced in the final part of this application.

A further, very clear indication of the effectiveness of the substance according to the invention could be determined when investigating the blood of these patients. To this end, the concentration of ammonia and ammonia compounds in the blood before and after the treatment was investigated, since—as explained in detail above—they are very probably the toxic active substance which is causal for the dying off in the brain cells, and therefore for the dementia of the patients.

In the investigation of the proportion of these substances, however, a massive problem is that they are very readily volatile. It is therefore necessary that the blood sample is deep frozen immediately after it is taken and only thawed out again immediately before the analysis. But also with this refinement of the measurement process, no statistically significant difference in the harmful substance content of the blood of Alzheimer patient and of a non-dementia comparison group could be determined.

It is to be assumed that the blood-brain barrier is a barrier that can only marginally passed through, if at all, by ammonia and the toxically active ammonia compounds. Only after the application of electromagnetic shock pulses to the patient's brain could a significant difference in the loading of the blood with ammonia and ammonia-containing compounds in dementia and in healthy patents be ascertained. The proportion of these harmful substances in the blood was 24-30 micromol per litre in non-loaded persons as well as in Alzheimer patients before treatment. After treatment with the active substance according to the invention, however, without the additional application of electromagnetic shock waves, the level of the harmful substances in the blood of Alzheimer's patients rose to as high as 350 micromol per litre. In the case of patients without dementia symptoms treated for comparison, this value after treatment remained unchanged at about 25-30 micromol per litre.

The clear increase of this contamination of the blood shows that such harmful substances from the brain cell must have been transported away and are no longer transferred into the blood and from there must be without hindrance degraded and disposed of by the existing circulations of the human body for the degradation of ammonia and ammonia compounds.

The absolute amount of harmful substances that causes devastating damage in the brain is a comparatively trivial disposal task for the blood circulation. The problem to date has been to transport the harmful substances out of the brain, via the blood-brain barrier into the blood circuit. This highly advantageous influence of the active substance according to the invention is the primary component during detoxification. The reinforcing effect of electromagnetic shock pulses is lower in proportion thereto, because it only occurs secondarily after prior active substance administration.

Since tests have shown that the effect of the electromagnetic shock pulses alone are not adequate. Only the combination of the active substance according to the invention with the application of shock pulses causes a thorough and rapid transport away of the harmful substances, which show the spectacular effects of the treatment within a week.

A possible explanation of the advantageous effect of electromagnetic radiation is that, with a high energy density of electromagnetic radiation in the affected body tissue, a significant change of the cell potential and possibly a heating is observed. In the skull, this heating can influence the blood-brain barrier and make it more permeable. Such effects are also demonstrated by the effect of heat sources on peripheral body parts (N. R. Saunders: Development of the blood-brain barrier to macromolecules. In: The Fluids and Barriers of the Eye and Brain, Editor: M. B. Segal, Verlag MacMillan, 1991, pp. 128-155. ISBN 0-849-37707-2).

Another advantageous effect of the N-(aminoacyl)-amino ester according to the invention is the treatment or prevention of blood flow disturbances. Positive effects are observed not only generally in the wide variety of known types of vascular insufficiency but in particular also in blood-flow disturbances in the cerebral area, such as the subcortical arteriosclerotic encephalopathy, also known as SAE or Binswanger's disease, stroke (apoplexy) and other cerebrovascular insufficiencies.

The following chain of effect is assumed: The N-(aminoacyl)-amino esters according to the invention pass through the blood-brain barrier, since, through the esterification according to the invention with arginine, ornithine and asparagine in the blood-brain barrier, suitable carrier transport media are present After this barrier has been overcome, a major part of the ester according to the invention are transformed back to N-(aminoacyl)-amino acid by hydrolysis in this region of the brain. Their blood-pressure lowering effect is known in principle. The increased release of dopamine and the simultaneous reduction of prolactin is significant. This effective could also be observed as expected in the experiments. After only half an hour, the prolactin level was significantly lowered, and decreased somewhat further during the following approximately three hours. An example is explained in the closing part.

In the experiments with the active substance according to the invention, a steep increase in the dopamine level could be observed as is known in principle from N-(aminoacyl)-amino acid, however without a simultaneous increase of noradrenaline and adrenaline. Until now, in the literature, in the context of a dopamine increase, an increase of the level of noradrenaline and adrenaline is generally always reported. A close relationship between these important neurotransmitters was thus always observed in the prior art: Active substances known in the prior art that have influenced the dopamine level have generally also changed adrenaline and noradrenaline in the same way. This phenomenon is known, for example, from drug dependents in which as a result of the administration of different drugs the level of dopamine rises clearly. An excessive increase of noradrenaline and adrenaline is always associated therewith, which massively damages the brain.

The effect of the substance according to the invention, which was not observed in the prior art, is that with a very clear dopamine increase the noradrenaline and adrenaline rise is only marginal. This effect is very essential for the effectiveness of the substance according to the invention: A physiological dopaminergic reaction occurs thereby, which is responsible for improving the blood flow and significantly improving the brain metabolism. The active substance according to the invention thus permits for the first time, by means of the stimulating substance dopamine, to intervene in the brain metabolism without an increase of noradrenaline or adrenaline simultaneously occurring.

The profile of the curve of dopamine increase determined here makes it obvious that the ester according to the invention is transformed back into an N-(aminoacyl)-amino acid by the body within a short time. It is to be assumed that the process is hydrolysis.

In other very interesting applications, it was found that the active substance according to the invention is also suitable for treating and/or for preventing a toxic loading of other organs, since the active substance according to the invention very generally exerts a detoxifying effect on the organ.

In a further embodiment, the invention presents the production of a medication for treating Alzheimer's disease and its secondary illnesses: Besides at least one of the active substances according to claim 1 or 7, it additionally contains arginine and/or ornithine and/or lysine and/or acetylcysteine or another cysteine and/or vitamin B1 and/or vitamin B6 and/or vitamin B12 and/or folic acid and/or vitamin D and/or the mineral potassium, for example as potassium citrate.

The potassium-derivative potassium citrate restores the ion equilibrium in the disposal of the Alzheimer toxin ammonia. It is displaced in that the Alzheimer-toxin ammonia is just as large as the potassium ion and therefore the mineral potassium is eliminated from the cell.

The deficiency in potassium causes a cellular acidosis and therefore an excess of acid ions or charge components, which hinder the enzyme function with a disturbed, non-physiological pH.

As a similar effect, it is known from intensive medicine that, with a decrease of the physiological pH of the blood from normally 7.38-7.42 to only 6.2, the patient dies within a short time and only immediate therapy can rescue his life.

In a similar way, with nerve cells that are faultily controlled for years by an over-acidic pH and which are overloaded with undisposed ammonia (Alzheimer toxin), the cellular damage is so great that it leads to the collapse of neurons and astrocytes.

An admixture of vitamin D is appropriate and necessary for the following reasons: In none of the investigated patients with Alzheimer's disease or with a pre-stressing by an elevated ammonia level, could a normal vitamin D level be ascertained. Numerous patients additionally suffered from osteoporosis. Because of the deficit of vitamin D, too little calcium was deposited in the bones. The excess of calcium was instead introduced into the brain cell, so that double damage occurred. The overloading of the brain cells with calcium initiates a nitroso stress or oxidative stress, which additionally damages the cells.

Overall, the nerve cells are damaged by ammonia and its effect as Alzheimer toxin, and by the side-effects occurring therewith, in four ways, namely by:

1. Damage and overloading of the nerve cell overall
2. Changes of the AMPA and NMDA receptors
3. The loss of potassium by retaining ammonium and
4. Disturbing the acid-base metabolism in the cell with a restriction of enzyme function by a pH outside 7.38 to 7.42.

With at least one of the active substances according to the invention, this disadvantageous effect can be counted by the following action mechanisms.

The formation of toxic plaques is prevented. The APP protein can no longer be cleaved at the sensitive points—the serine. The beta and gamma secretase is no longer activated, which are responsible for forming the insoluble plaques.

Furthermore, the toxic amino acid homocysteine is reduced, which prevents the aggressive damage of blood vessels and activates the necessary enzyme unction in the cell.

Possible therapies are presented below based on the substances described above and their effect: It was already mentioned above that the N-(aminoacyl)-amino compounds according to the invention in combination with electromagnetic shock waves permit a significant improvement of the evidence of ammonia and ammonia compounds in the blood in dementia patients. For this purpose, the invention provides a highly interesting diagnosis plan. In the first step, the body part to be examined or the body region to be examined is subjected to electromagnetic shock pulses. In the second step, a blood sample is taken and then immediately centrifuged as a third step. In the fourth step, the plasma is investigated for ammonia and ammonia compounds.

If the blood examination is cannot be performed directly after the sampling, it as proven practicable that the plasma is deep frozen after the third step and only thawed again immediately before the fourth step.

A very essential advantage of this diagnostic plan according to the invention is the possibility of a preventive examination. Since the N-(aminoacyl)-amino ester according to the invention together with the electromagnetic shock waves effect a very dramatic increase of the amount of excreted ammonia compounds and ammonia, a preventive examination is thereby also possible. Already when the problems in the metabolism of the brain cell begin to be manifested, but the amount of ammonia and ammonia compounds generated in the cell in the process is still tolerable, this examination gives a first indication, so that a corresponding treatment can be started.

When the medication therapy starts at a—very early—time in the progress of Alzheimer's disease, in which the dementia symptoms do not yet appear as relevant, and have therefore remained unnoticed, a purely medication treatment already ensures sufficient transport away of the harmful substances. The somewhat more elaborate treatment with electromagnetic shock waves could be eliminated in such a variant.

In very serious cases or cases recognized at a very late stage, the purely medication therapy can be supplemented with a therapy with electromagnetic shock waves, which takes place a short time before the administration of the medication. A suitable therapy plan provides for a daily treatment duration with interrupting electromagnetic shock pulses for maximum one half hour. Then, the provided dose of the respectively provided N-(aminoacyl)-amino ester according to the invention is administered. The treatment is continued on the next day and extends until a duration of about one month maximum.

Further details and features of the invention are explained below in greater detail with reference to examples. However, they are not intended to limit the invention but only explain it. In schematic view:

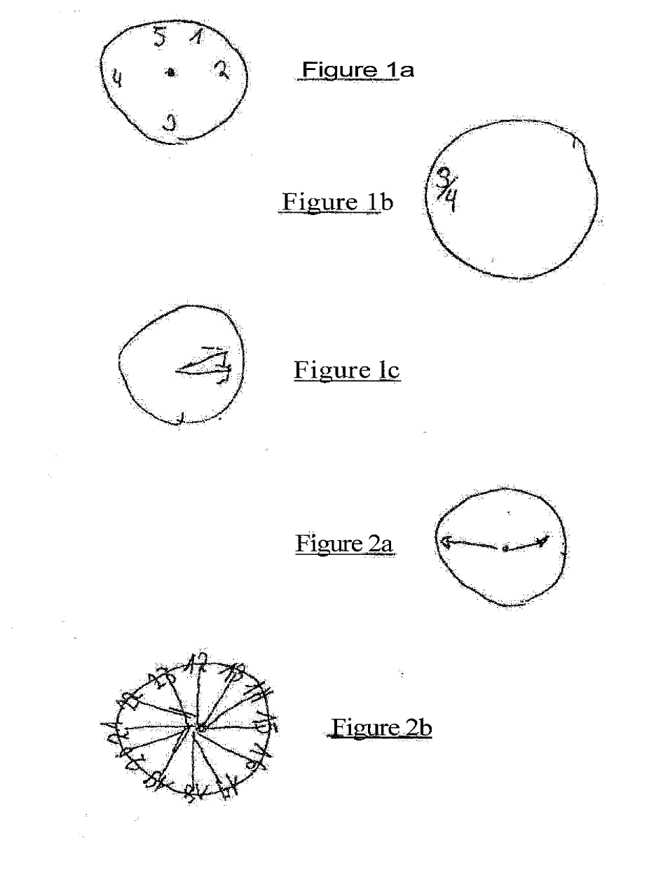

FIGS. 1a-1c show a clock-drawing test of an Alzheimer patient before treatment

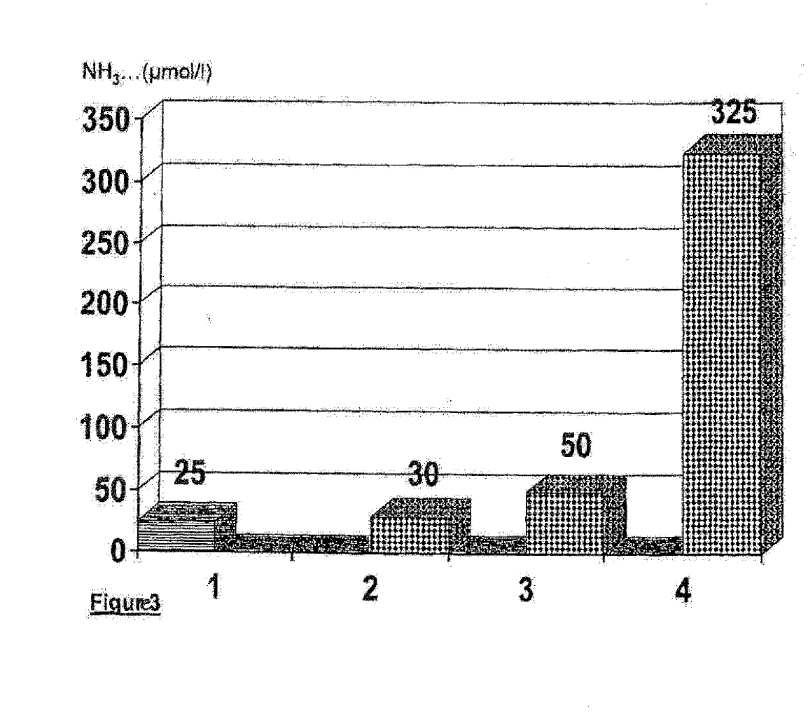

FIGS. 2a-2c show a clock-drawing test as above but after treatment

FIG. 3 shows a bar chart of the components of ammonia and ammonia compounds in the blood of Alzheimer patients

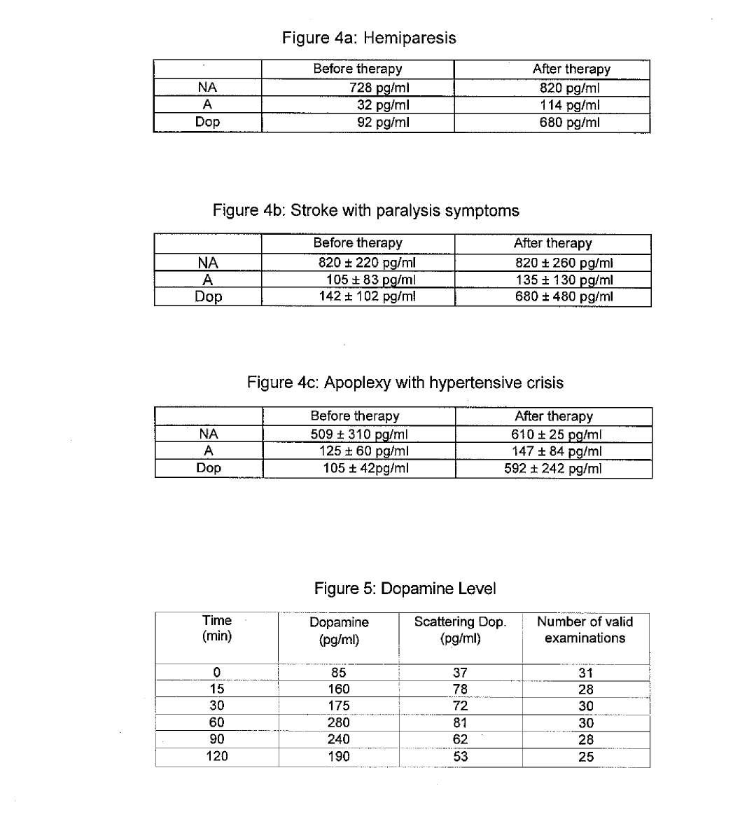

FIG. 4a shows noradrenaline, adrenaline and dopamine with hemiparesis before and after therapy

FIG. 4b as above but with acute stroke with various symptoms of paralysis

FIG. 4c as above but with apoplexy with hypertensive crisis

FIG. 5 shows dopamine level in the blood of an Alzheimer patients after treatment according to the invention

In detail, the figures show:

FIGS. 1a-1c show the test result for an Alzheimer patient in the clock drawing test before treatment. In FIG. 1a, the patient had been set the task of drawing a clock face in a given circle. It can be seen that he was not capable of positioning the numerals of a clock face reasonably in sequence and number.

In FIGS. 1 b and 1c, he had been set the task of drawing the position of the hands for a “quarter to three” (14:45) in a given circle. FIG. 1 b shows as result the entry of “three quarters” shown as a number. Nevertheless, the position of the entry of this number corresponds to the actually correct position of the minute hand. However, an hour hand is completely missing. In FIG. 1c, an hour hand is drawn in an approximately meaningful position, the minute hand, however, is completely wrongly positioned.

In FIGS. 2a and 2b, the test result after one-week's treatment with the active substance according to the invention is drawn in each case. In FIG. 2a, the patient has shown the correct position of the hour hand and minute hand for the time “quarter to three”. FIG. 2b shows that he was able to draw a correctly subdivided clock face, in this case even with the appropriate designations for the hours of the afternoon.

In FIG. 3, as bar chart, the proportions of ammonia and ammonia compounds in the blood of Alzheimer patients are plotted before and after treatment with the N-(aminoacyl)-amino ester according to the invention in comparison to the values of healthy comparison persons are drawn.

The first value, with approx. 25 micromol per liter—bar 1, is the mean value for the proportion of ammonia, ammonium and ammonium derivatives in the blood of comparison persons. After administration of the active substance according to the invention to the comparison persons, no effect could be observed.

In the case of patients with Alzheimer's disease, the proportion without any treatment is only slightly higher, namely approx. 30 micromol per litre—bar 2. The increase indicates that these toxic substances occur to a greater extent. However, an exact diagnosis with the aid of this value is not possible, since the actual concentration in the brain cells cannot be derived from the proportion in the blood, as the blood-brain barrier keeps the greatest proportion of these toxically acting substances back and thereby “falsifies” the measurement drastically.

In the right half of the bar chart, the result of the treatment with the medication according to the invention is recorded. The blood of Alzheimer patients that were treated solely by means of the medication contains on average a harmful substance proportion of about 50 micromol per liter—bar 3. The level, which is about ⅔ higher than that of untreated Alzheimer patients makes clear, that under the effect of the active substance according to the invention, more toxin is released from the brain cells.

Under treatment not only with the medication but additionally also the exposure to an electromagnetic shock source, the proportion of the toxin in the blood has risen very drastically, namely to a level of about 325 micromoles per litre, that is to say almost eleven-fold—bar 4.

This drastic increase is, with some probability, induced by the fact that the permeability of the blood-brain barrier for the harmful substances under the influence of the electromagnetic shock waves is significantly increased and thereby the output of the toxic substances from the affected cells can be increased.

The FIGS. 4a to 4c show the level of the important neurotransmitter noradrenaline (NA) and adrenaline (A) and dopamine (Dop) before the therapy and after the therapy, in each case with patients with a stroke together with a massive crisis in each case. An active substance according to the invention was administered intravenously in each case, specifically shortly after the occurrence of the critical state in each case.

FIG. 4a shows the measurement values for a patient who suffered from a severe hemiparesis following a stroke. The measurement values for noradrenaline (NA) from 728 picograms per millilitre previously have increased to only 820 picograms per millilitre after the therapy. The adrenaline level has risen from 32 pg/ml previously to 114 pg/ml. However the dopamine content of the blood has increased very dramatically from only 92 pg/ml very dramatically to 680 pg/ml.

FIG. 4 b shows the measurement values of a patient in whom the stroke had caused clear paralysis symptoms. The level of noradrenaline increased from 820 pg/ml only slightly to 880 pg/ml and also the adrenaline level increased from 105 pg/ml to only 135 pg/ml. However, the dopamine level changed very drastically from 142±102 pg/ml previously to an impressive 680±480 pg/ml.

A similar measurement result is shown in FIG. 4 c for a patient whose apoplexy was accompanied by a hypertensive crisis.

The three examples of FIGS. 4a-4c make it clear that it has been possible for the first time here to name an active substance that has a dopaminergic effect and can unfold this beneficial effect even through the blood-brain barrier within the brain and which nevertheless effects the former disadvantageous increase of the noradrenaline level and adrenaline level always associated with the dopamine increase.

In FIG. 5, from a patient who has been intravenously administered with an active substance according to the invention, the values measured thereby for his dopamine level are recorded in dependence on time. In FIG. 5, it can be clearly seen that his dopamine level has increased to about threefold after only an hour. It can also be recognised that the rise is relatively very much steeper than the lowering, which takes place after about 1½ hours. The advantageous effect of the dopamine thus lasts for a relatively long time.

In the third column, the scattering of the dopamine values in pg/ml for the same time frame. It can be seen that these values, too, reach their maximum after a time of 60 minutes. However, they are only increased by a comparatively low 20%, which is virtually negligible in contrast to the increase of the dopamine by 230% which is measured here.

For the sake of control, the respective number of valid investigated patients is given in the fourth column. In practice, it is unavoidable that errors occur in the course of some measurements, such as an interruption in the cooling chain of the samples. If, with some probability, such a problem or a similar one has occurred, scientific care is required not to evaluate this series of measurements. The number of valid investigated patients is therefore not the same for each time section.

### LIST OF REFERENCE CHARACTERS

Ar N-(aminoacyl)amino arginate ester
As N-(aminoacyl)-amino asparaginate ester
Or N-(aminoacyl)-amino ornithate ester
A Adrenaline level in the blood
Dop Dopamine level in the blood
NA Noradrenaline level in the blood

1. N-(aminoacyl)-amino compound, represented by the following formula 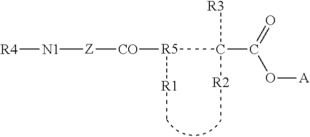
Wherein
R1 denotes hydrogen, low alkyl or carbonyl, and
N1 denotes an NH group and
R2 denotes hydrogen or low alkylphenyl or aralkyl or imidazoalkyl or indolylalkyl, which may be substituted by low alkyl or hydroxy or alkylhydroxy or methylenedioxy or mercapto or alkylmercapto or amino or guanandino or carboxa and
R1 and R2 together may complete a pyrrolidine or piperidine or thiazolidine ring and each ring can be substituted by a low alkyl or aralkyl or phenyl or furyl or thienyl or pyridyl or naphthyl, which in turn may be substituted by a low alkyl or hydroxy low alkyl or mercapto low alkyl or hydroxy or low alkoxy or alkylene dioxy or halogen or nitro or amino or low alkylamino or acylamino and
R3 denotes hydrogen or methyl or low alkyl and R4 denotes hydrogen or alkyl or the group remaining on exclusion of R4 from the formula and
Z is a straight chain or branched alkylene, which may contain up to 3 carbon atoms. and R5 is nitrogen or sulphur or oxygen or salts thereof and ester compounds, characterised in that A is an ester or amino acid.

2. N-(aminoacyl)-amino compound according to claim 1, characterized in that A is an arginate ester or an ornithate ester or an asparaginate ester.

3. N-(aminoacyl)-amino compound according to claim 2, characterized in that it consists of a mixture of N-(aminoacyl)-amino arginate ester (Ar) and/or N-(aminoacyl)-amino ornithate ester (Or) and/or N-(aminoacyl)-amino asparaginate ester (As).

4. N-(aminoacyl)-amino compound according to claim 3, characterized in that the proportion of (Ar) is greater than the proportion of (Or) and of (As).

5. N-(aminoacyl)-amino compound according to claim 4, characterized in that the proportion of (Ar) constitutes at least 50%.

6. N-(aminoacyl)-amino compound according to claim 5, characterized in that the proportions of (Or) and of (As) are approximately equally sized.

7. N-(aminoacyl)-amino compound according to claim 1, characterized in that A is a basic amino acid.

8. N-(aminoacyl)-amino compound according to claim 7, characterized in that A is
asparagine aspartate and/or
arginine aspartate and/or
arginine lysine and/or
cysteine.

9. N-(aminoacyl)-amino salt, represented by the following formula 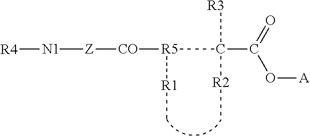
wherein
R1 denotes hydrogen, low alkyl or carbonyl, and N1 denotes an NH group, and
R2 denotes hydrogen or low alkylphenyl or aralkyl or imidazolylalkyl or indolylalkyl, which may be substituted by low alkyl or hydroxy or alkylhydroxy or methylenedioxy or mercapto or alkylmercapto or amino or guanidino or carboxa and
R1 and R2 together can complete a pyrrolidine or piperidine or thiazolidine ring and each ring may be substituted by a low alkyl or aralkyl or phenyl or furyl or thienyl or pyridyl or naphthyl, which in turn can be substituted by a low alkyl or hydroxy low alkyl or mercapto low alkyl or hydroxy or low alkoxy or alkylene dioxy or halogen or nitro or amino or low alkylamino or acylamino and
R3 denotes hydrogen or methyl or low alkyl and R4 represents hydrogen or alkyl or the group remaining with the exclusion of R4 from the formula and
Z is a straight-chain or branched alkylene, which may contain up to 3 carbon atoms and
R5 is nitrogen or sulphur or oxygen or salts thereof and ester compounds, characterized in that
A is a sodium or a potassium salt of arginate and/or of ornithate and/or of asparaginate, that is to say a sodium arginate and/or a potassium arginate and/or a sodium ornithate and/or a potassium ornithate and/or a sodium asparaginate and/or a potassium asparaginate.

10. Use of N-(aminoacyl)-amino compounds or N-(aminoacyl)-amino salts according to claim 1 for producing pharmaceutical preparations and medications.

11. Use of N-(aminoacyl)-amino compounds or N-(aminoacyl)-amino salts according to claim 1 for producing pharmaceutical preparations and medications for treating and/or for preventing toxic loadings by ammonia and/or ammonia compounds.

12. Use of N-(aminoacyl)-amino compounds or N-(aminoacyl)-amino salts according to claim 1 for producing pharmaceutical preparations and medications for treating and/or for preventing Alzheimer's disease.

13. Use of N-(aminoacyl)-amino compounds or N-(aminoacyl)-amino salts according to claim 1 for producing pharmaceutical preparations and medications for treating and/or for preventing blood flow disorders such as vascular insufficiencies and hypertension and in blood flow disorders in the cerebral area, such as subcortical arteriosclerotic encephalopathy, also known as SAE or Binswanger's disease or stroke (apoplexy) or Parkinson's disease or other cerebrovascular insufficiencies.

14. Use of N-(aminoacyl)-amino compounds or N-(aminoacyl)-amino salts according to claim 1 for producing pharmaceutical preparations and medications for producing a dopaminergic effect.

15. Use of N-(aminoacyl)-amino compounds or N-(aminoacyl)-amino salts according to claim 1 for producing pharmaceutical preparations and medications for treating and/or for preventing toxic overloads of particular organs.

16. Method of producing a medication for treating Alzheimer's disease and its secondary diseases according to one of the preceding claims, characterized in that it contains at least one active substance according to claim 1 and additionally
arginine and/or
ornithine and/or
asparagine and/or
acetylcysteine or another cysteine and/or
vitamin B1 and/or
vitamin B8 and/or
vitamin B12 and/or
folic acid and/or
vitamin D and/or
the mineral potassium, for example as potassium citrate.

17. Diagnosis plan required for use of N-(aminoacyl)-amino compounds or N-(aminoacyl)-amino salts according to claim 1 characterized in that
in the first step, the body part to be examined or the body region to be examined is subjected to electromagnetic shock pulses, and immediately thereafter
in the second step, a blood sample is taken and immediately thereafter
in the third step, the blood sample is centrifuged and immediately thereafter
in the fourth step, the plasma is investigated for ammonia and ammonia compounds.

18. Diagnosis plan according to claim 17, characterized in that, after the third step, the plasma is deep frozen and only thawed out again immediately before the fourth step.

19. Therapy plan for application of N-(aminoacyl)-amino compounds or N-(aminoacyl)-amino salts according to claim 1, characterized in that the body part to be treated or the body area to be treated is subjected to a multiplicity of interrupting electromagnetic shock impulses for a time of up to maximum approximately one half hour and thereafter the provided dose of the respective N-(aminoacyl)-amino compound administered in each case, and this treatment is continued on the next day for up to a duration of maximum approximately one month.

20. Method of producing a medication for treating Alzheimer's disease and its secondary diseases according to claim 7, characterized in that it contains at least one active substance according to claim 1 and additionally
arginine and/or
ornithine and/or
asparagine and/or
acetylcysteine or another cysteine and/or
vitamin B1 and/or
vitamin B8 and/or
vitamin B12 and/or
folic acid and/or
vitamin D and/or
the mineral potassium, for example as potassium citrate.
